(19) United States
(12) Patent Application Publication
(10) Pub. No.: US 2009/0234233 A1
(43) Pub. Date: Sep. 17, 2009

(54) ULTRASONIC ENDOSCOPE

(75) Inventors: Kazuhiko NAGANO, Kaisei-machi (JP); Hiroaki Hyuga, Kaisei-machi (JP); Atsushi Osawa, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/401,279

(22) Filed: Mar. 10, 2009

(30) Foreign Application Priority Data
Mar. 13, 2008 (JP) 2008-064388
Jul. 29, 2008 (JP) 2008-195485

(51) Int. Cl. A61B 8/14

(52) U.S. Cl. 600/462

(57) ABSTRACT

In an ultrasonic endoscope, excessive temperature rise due to heat generated from ultrasonic transducers and/or an image pickup device is prevented. The ultrasonic endoscope includes: an ultrasonic transducer part including plural ultrasonic transducers for transmitting and receiving ultrasonic waves, and a backing material provided on a back of the plural ultrasonic transducers and having plural signal terminals provided on a surface opposite to the plural ultrasonic transducers; a signal line holding part including a highly heat conducting filler filling a space holding a group of shield lines electrically connected to the ultrasonic transducers via the plural signal terminals, and coupled to the backing material; and a highly heat conducting layer provided in contact with the signal line holding part, and thereby coupled to the signal line holding part.

### BACKGROUND OF THE INVENTION

1. Field of the Invention

The present invention relates to a heat dissipation structure of an ultrasonic endoscope including an ultrasonic probe to be used for body cavity examination of upper digestive organs, bronchial tube, and so on.

2. Description of a Related Art

In medical fields, various imaging technologies have been developed in order to observe the interior of an object to be inspected and make diagnoses. Among them, especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but also gynecology, circulatory system, digestive system, etc.

The ultrasonic imaging is an image generation technology utilizing the nature of ultrasonic waves that the waves are reflected at a boundary between regions having different acoustic impedances (e.g., a boundary between structures). Typically, an ultrasonic diagnostic apparatus is provided with a body surface ultrasonic probe to be used in contact with the object or intracavity ultrasonic probe to be used by being inserted into a body cavity of the object. Further, in recent years, an ultrasonic endoscope in combination of an endoscope for optically observing the interior of the object and an ultrasonic probe for intracavity has been used.

Ultrasonic beams are transmitted toward the object such as a human body and ultrasonic echoes generated in the object are received by using the ultrasonic endoscope, and thereby, ultrasonic image information is acquired. On the basis of the ultrasonic image information, ultrasonic images of structures (e.g., internal organs, diseased tissues, or the like) existing within the object are displayed on a display unit of an ultrasonic endoscopic apparatus main body connected to the ultrasonic endoscope.

As an ultrasonic transducer for transmitting and receiving ultrasonic waves, a vibrator (piezoelectric vibrator) having electrodes formed on both sides of a material that expresses a piezoelectric property (a piezoelectric material) is generally used. As the piezoelectric material, piezoelectric ceramics represented by PZT (Pb (lead) zirconate titanate), a polymeric piezoelectric material represented by PVDF (polyvinylidene difluoride), or the like is used.

When a voltage is applied to the electrodes of the vibrator, the piezoelectric material expands and contracts due to the piezoelectric effect to generate ultrasonic waves. Accordingly, plural vibrators are one-dimensionally or two-dimensionally arranged and the vibrators are sequentially driven, and thereby, an ultrasonic beam to be transmitted in a desired direction can be formed. Further, the vibrators expand and contract by receiving propagating ultrasonic waves to generate electric signals. These electric signals are used as reception signals of the ultrasonic waves.

When ultrasonic waves are transmitted, drive signals having great energy are supplied to the ultrasonic transducers. In this regard, not the entire energy of the drive signals is converted into acoustic energy but a significant proportion of the energy becomes heat, and there has been a problem that the temperature rises in use of the ultrasonic endoscope. However, the insertion part of the ultrasonic endoscope is used in direct contact with the living body such as a human body, and there has been made a request that the surface temperature of the insertion part of the ultrasonic endoscope is controlled to a predetermined temperature or less.

As a related technology, Japanese Patent Application Publication JP-A-9-140706 (“Probe of Ultrasonic Diagnostic Apparatus”) discloses a probe including heat collecting means for collecting heat of the probe in an interior of the probe and heat transfer means for guiding the heat collected by the heat collecting means to a location apart from a heat source opposite to the interior of the probe. However, in a small-diameter endoscope having an outer diameter of 5 mm to 6.9 mm as a bronchial tube endoscope, it is difficult to provide the heat transfer means like a heat pipe disclosed in JP-A-9-140706 within the endoscope tube. Further, even if the heat pipe can be provided within the endoscope tube, there is a problem that the sectional area of the heat pipe becomes smaller and the sufficient heat dissipation effect is not obtained in the small-diameter endoscope as a bronchial tube endoscope.

Japanese Patent Application Publication JP-P2006-204552A (“Ultrasonic Probe”) and Japanese Registered Utility Model JP-Z-3061292 (“Ultrasonic Transducer Structure”) disclose a structure for releasing the heat from a vibrator to a shield case or signal cable. In the case of the ultrasonic probe for body surface as embodiments of JP-P2006-204552A and JP-Z-3061292, although the size of the cable or the like is large enough to secure heat dissipation performance, if the same structure is used for an ultrasonic endoscope as it is, its size is too large to form an endoscope having a small diameter.

In the case of an ultrasonic endoscope, the shield lines for signal transmission have smaller diameters and high heat resistance, and thus, the heat dissipation performance cannot be secured. On the other hand, a shield foil on the outer periphery for covering plural signal lines has a relatively large sectional area, but it is connected to the ground of the system, and accordingly, there is a problem, when a heat transfer material (a copper foil or the like) electrically continuous with the ultrasonic transducers is connected to the shield foil on the outer periphery, the noise at the system side mixes in the reception signals. Furthermore, when the shield foil is attached to the outer periphery of the plural signal lines, the outer circumference of the entire cable becomes thick, and accordingly, there is a problem that the cable can not be provided within a small-diameter tube (having an inner diameter of 5.9 mm or less) as a bronchial tube endoscope.

Regarding an ultrasonic probe for body surface, technological innovations progress towards improvements in transmission performance by multilayered configuration of the piezoelectric element for higher diagnostic accuracy. However, the oscillation output of ultrasonic waves is increased by the multilayered configuration of the piezoelectric element, the amount of heat radiation becomes larger, and accordingly, there is a problem that the temperature of the part in contact with the inner wall of the body cavity may excessively rise in the conventional structure. Furthermore, in the case of a small endoscope including an image pickup device (CCD), the temperature of the leading end of the endoscope may excessively rise due to heat generation by the CCD.

On the other hand, regarding an ultrasonic endoscope, downsizing is needed. Especially, as an ultrasonic endoscope for bronchial tube having a strong need for downsizing, a small-diameter endoscope having an outer diameter of 6.9 mm is used and even smaller diameter is needed.

### SUMMARY OF THE INVENTION

The present invention has been achieved in view of the above-mentioned problems. A purpose of the present invention is to prevent excessive temperature rise due to heat generated from ultrasonic transducers and/or an image pickup device in an ultrasonic endoscope.

In order to accomplish the purpose, an ultrasonic endoscope according to one aspect of the present invention includes: an ultrasonic transducer part including plural ultrasonic transducers for transmitting and receiving ultrasonic waves, and a backing material provided on a back of the plural ultrasonic transducers and having plural signal terminals provided on a surface opposite to the plural ultrasonic transducers; a signal line holding part including a highly heat conducting filler filling a space holding a group of shield lines electrically connected to the plural ultrasonic transducers via the plural signal terminals, and coupled to the backing material; and a highly heat conducting layer provided in contact with the signal line holding part, and thereby coupled to the signal line holding part.

According to the present invention, since the signal line holding part under the backing material provided on the back of the ultrasonic transducers is filled with the highly heat conducting filler and further the highly heat conducting layer is provided in contact with the signal line holding part in the ultrasonic endoscope, the heat release performance of the small-diameter endoscope probe can be improved and the excessive temperature rise due to heat generated from the ultrasonic transducers and/or the image pickup device can be prevented. Thereby, the output or reception sensitivity of ultrasonic transducers can be increased and an ultrasonic diagnostic apparatus with high diagnostic accuracy can be realized.

### BRIEF DESCRIPTION OF THE DRAWINGS

FIG. 1 is a schematic diagram showing an appearance of an ultrasonic endoscope according to the respective embodiments of the present invention;

FIG. 2 shows an ultrasonic endoscopic apparatus including the ultrasonic endoscope according to the respective embodiments of the present invention and an ultrasonic endoscopic apparatus main body;

FIG. 3 is a side sectional view schematically showing the leading end of the insertion part of the ultrasonic endoscope according to the first embodiment of the present invention;

FIG. 4 is a front sectional view along A-Al in FIG. 3;

FIG. 5 is a perspective view showing a conceptual structure of multilayered piezoelectric element according to the first embodiment;

FIG. 6 is a sectional view showing an ultrasonic transducer part to which the multilayered piezoelectric element shown in FIG. 5 is applied;

FIG. 7 is a perspective view showing a highly heat conducting layer in the first embodiment of the present invention;

FIG. 8 shows a first modified example of the first embodiment of the present invention;

FIG. 9 shows a second modified example of the first embodiment of the present invention;

FIG. 10 shows a third modified example of the first embodiment of the present invention;

FIG. 11 is a front sectional view of the leading end of the insertion part of the ultrasonic endoscope according to the second embodiment of the present invention;

FIG. 12 is a perspective view showing a highly heat conducting layer and shield foils in the second embodiment of the present invention;

FIG. 13 is a front sectional view of the leading end of the insertion part of the ultrasonic endoscope according to the third embodiment of the present invention;

FIG. 14 is a perspective view showing a highly heat conducting layer and shield foils in the third embodiment of the present invention;

FIG. 15 is a side sectional view schematically showing the leading end of the insertion part of the ultrasonic endoscope according to the fourth embodiment of the present invention;

FIG. 16 is a perspective view showing a highly heat conducting layer in the fourth embodiment of the present invention;

FIG. 17 is a perspective view showing a highly heat conducting layer and shield foils in the fifth embodiment of the present invention;

FIG. 18 is a perspective view showing a highly heat conducting layer and shield foils in the sixth embodiment of the present invention;

FIG. 19 is a side sectional view schematically showing the leading end of the insertion part of the ultrasonic endoscope according to the seventh embodiment of the present invention;

FIG. 20 is a perspective view showing a highly heat conducting layer in the seventh embodiment of the present invention;

FIG. 21 shows a relationship between the coefficient of thermal conductivity of a filler and the surface temperature rise;

FIG. 22 shows a relationship between the coefficient of thermal conductivity of the highly heat conducting layer and the surface temperature rise; and

FIG. 23 shows a relationship between the thickness of the highly heat conducting layer and the surface temperature rise.

### DESCRIPTION OF THE PREFERRED EMBODIMENTS

Hereinafter, embodiments of the present invention will be explained in detail with reference to the drawings. The same reference numbers will be assigned to the same component elements and the description thereof will be omitted.

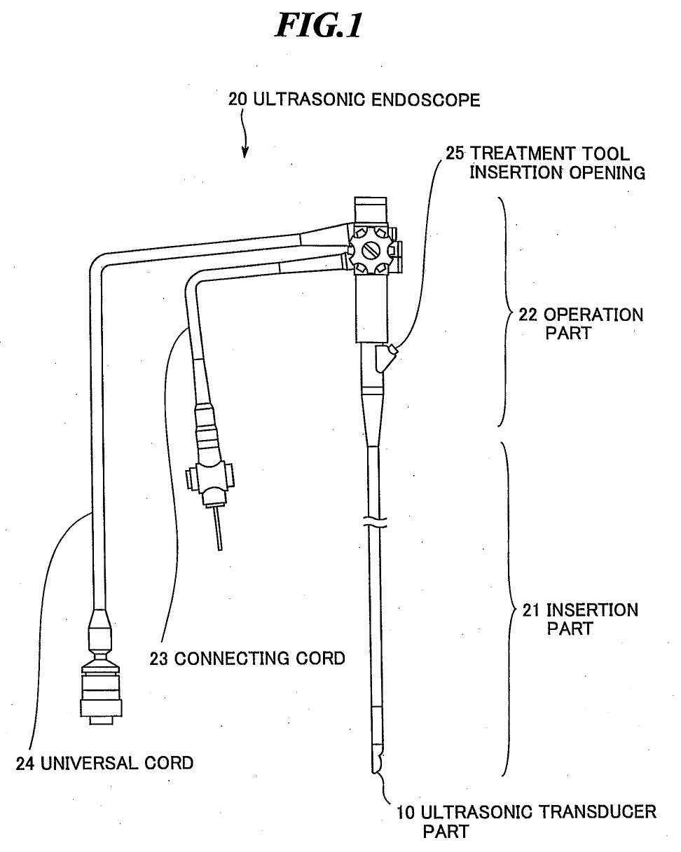

FIG. 1 is a schematic diagram showing an appearance of an ultrasonic endoscope according to the respective embodiments of the present invention. As shown in FIG. 1, an ultrasonic endoscope 20 includes an insertion part 21, an operation part 22, a connecting cord 23, and a universal cord 24. The insertion part 21 includes an elongated tube formed of a member having flexibility for insertion into the body of an object to be inspected, and an ultrasonic transducer part 10 at the leading end thereof.

The operation part 22 is provided at the base end of the insertion part 21 and connected to an ultrasonic endoscopic apparatus main body via the connecting cord 23 and the universal cord 24. A treatment tool insertion opening 25 provided in the operation part 22 is a hole for leading in a treatment tool such as a punctuation needle or forceps. Various treatments are performed within a body cavity of the object by operating it with the operation part 22.

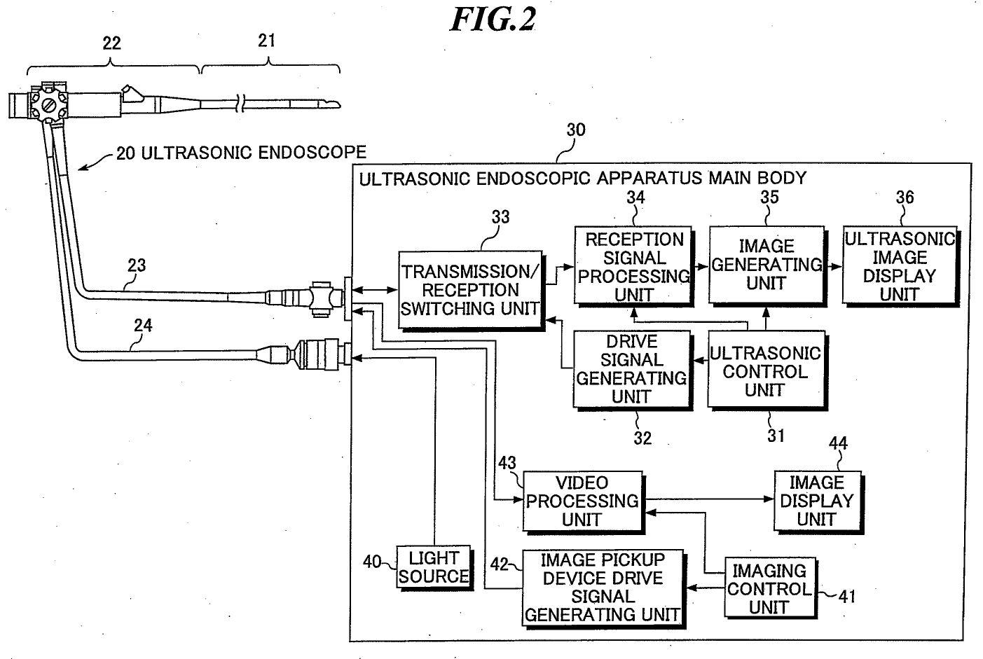

FIG. 2 shows an ultrasonic endoscopic apparatus including the ultrasonic endoscope according to the respective embodiments of the present invention and an ultrasonic endoscopic apparatus main body. The plural ultrasonic transducers included in the ultrasonic transducer part 10 are electrically connected to the ultrasonic endoscopic apparatus main body 30 by the plural shield lines via the insertion part 21, the operation part 22, and the connecting cord 23. Those shield lines transmit plural drive signals generated in the ultrasonic endoscopic apparatus main body 30 to the respective ultrasonic transducers and transmit plural reception signals outputted from the respective ultrasonic transducers to the ultrasonic endoscopic apparatus main body 30.

The ultrasonic endoscopic apparatus main body 30 includes an ultrasonic control unit 31, a drive signal generating unit 32, a transmission/reception switching unit 33, a reception signal processing unit 34, an image generating unit 35, an ultrasonic image display unit 36, a light source 40, an imaging control unit 41, an image pickup device drive signal generating unit 42, a video processing unit 43, and an image display unit 44.

The ultrasonic control unit 31 controls imaging operation using the ultrasonic transducer part 10. The drive signal generating unit 32 includes plural drive circuits (pulsers or the like), for example, and generates plural drive signals to be used for respectively driving the plural ultrasonic transducers. The transmission/reception switching unit 33 switches between output of the drive signals to the ultrasonic transducer part 10 and input of the reception signals from the ultrasonic transducer part 10.

The reception signal processing unit 34 includes plural preamplifiers, plural A/D converters and a digital signal processing circuit or CPU, for example, and performs predetermined signal processing such as amplification, phasing addition, and detection on the reception signals to be outputted from the plural ultrasonic transducers. The image generating unit 35 generates image data representing ultrasonic images based on the reception signals on which the predetermined signal processing has been performed. The ultrasonic image display unit 36 displays the ultrasonic images based on the image data generated in this manner.

The light source 40 emits light used for illumination of the object. The light outputted from the light source 40 illuminates the object via the universal cord 24 through an illumination window of the insertion part 21. The illuminated object is imaged by an image pickup device part (not shown) through an observation window of the insertion part 21, and video signals outputted from the image pickup device part are inputted to the video processing unit 43 of the ultrasonic endoscopic apparatus main body 30 via the connecting cord 23.

The imaging control unit 41 controls imaging operation using the image pickup device part. The image pickup device drive signal generating unit 42 generates drive signals for driving the image pickup device part. The video processing unit 43 generates image data based on the video signals to be inputted from the image pickup device part. The image display unit 44 inputs the image data from the video processing unit 43 and displays images of the object.

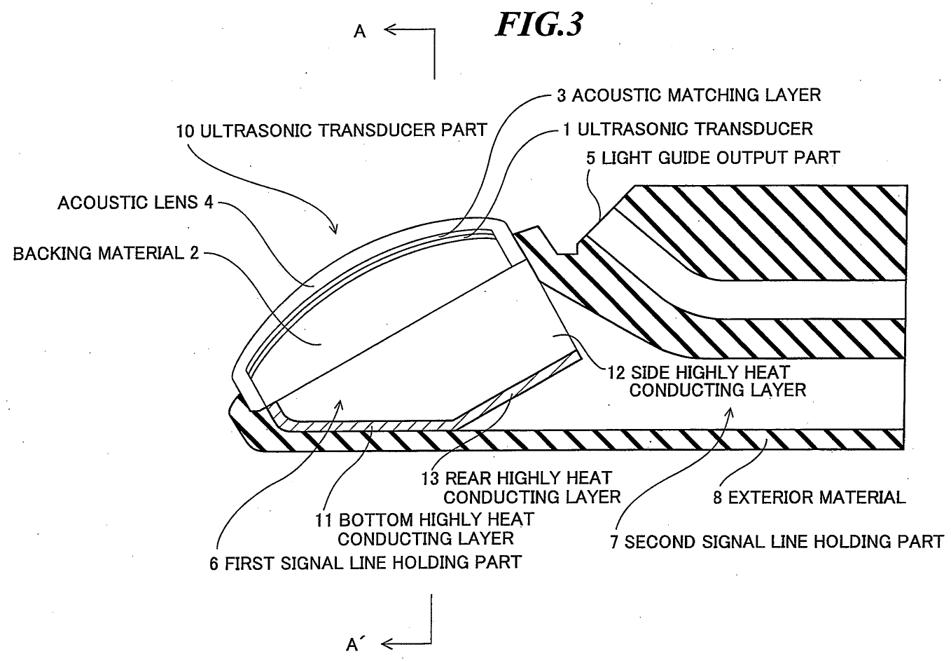

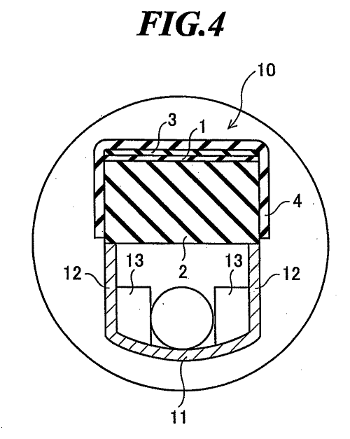

FIG. 3 is a side sectional view schematically showing the leading end of the insertion part of the ultrasonic endoscope according to the first embodiment of the present invention. Further, FIG. 4 is a front sectional view along A-A′ in FIG. 3. As shown in FIGS. 3 and 4, the insertion part of the ultrasonic endoscope has plural (e.g., 64) ultrasonic transducers 1 for transmitting and receiving ultrasonic waves, a backing material 2 for supporting the plural ultrasonic transducers 1, an acoustic matching layer 3 for providing match of acoustic impedances between the plural ultrasonic transducers 1 and the object, an acoustic lens 4 for focusing ultrasonic waves in an elevation direction perpendicular to the arrangement direction (azimuth direction) of the ultrasonic transducers 1, a light guide output part 5 for outputting light, the image pickup device part (not shown) for optically imaging an affected part, and an exterior material 8 covering the respective parts.

The structure of the piezoelectric element forming the ultrasonic transducer 1 is basically a single-layer structure in which electrodes are formed on both sides of one piezoelectric material, and a multilayered piezoelectric element in which plural piezoelectric materials and plural electrodes are alternately stacked is also used because of microfabrication and integration with the recent developments of MEMS (micro electro mechanical systems) related devices. In the piezoelectric element, the capacitance of the entire piezoelectric element can be increased by connecting the electrodes for applying electric fields to plural piezoelectric material layers in parallel. Accordingly, even when the size of the piezoelectric element is made smaller, the rise in electric impedance can be suppressed.

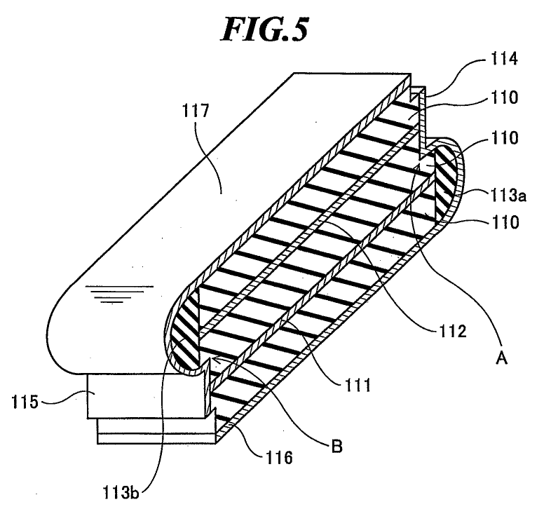

FIG. 5 is a perspective view for conceptual explanation of the structure of an example of multilayered piezoelectric element. The multilayered piezoelectric element 100 has a multilayered structure in which three piezoelectric material layers 110 and a first internal electrode 111 and a second internal electrode 112 are alternately stacked, a side insulating film 113a formed on one side surface of the multilayered structure (on the right in the drawing), a side insulating film 113b formed on the other side surface of the multilayered structure (on the left in the drawing), side electrodes 114 and 115, a lower electrode 116, and an upper electrode 117. Although three piezoelectric material layers 110 are shown in FIG. 5, the number of piezoelectric material layers may be four or more. When the number of piezoelectric material layers is five or more, the multilayered structure includes plural first internal electrodes 111 and plural second internal electrodes 112.

As shown in FIG. 5, since steps A and B are formed on both side surfaces of the multilayered structure, the right end of the internal electrode 111 is located on the convex portion of the right side surface, and the left end of the internal electrode 112 is located on the convex portion of the left side surface. Therefore, the side insulating films 113a and 113b are easily formed. The side insulating film 113a covers the right end of the internal electrode 111 in the convex portion of the right side surface of the multilayered structure, and the side insulating film 113b covers the left end of the internal electrode 112 in the convex portion of the left side surface of the multilayered structure. Since each of the side insulating films 113a and 113b has a shape including a part of a cylinder or a curved surface, the side electrodes 114 and 115 are easily formed but hardly cut.

Here, the side electrodes 114 and 115 and the lower electrode 116 and the upper electrode 117 may simultaneously or separately be formed. In either case, the side electrode 114 is connected to the lower electrode 116 and the second internal electrode 112 as odd-numbered electrodes (the first group of electrodes) and insulated from the first internal electrode 111 and the upper electrode 117 as even-numbered electrodes (the second group of electrodes that do not belong to the first group of electrodes). Further, the side electrode 115 is connected to the first internal electrode 111 and the upper electrode 117 as the even-numbered electrodes (the second group of electrodes that do not belong to the first group of electrodes) and insulated from the lower electrode 116 and the second internal electrode 112 as the odd-numbered electrodes (the first group of electrodes). When a voltage is applied between the lower electrode 116 and the upper electrode 117, electric fields are applied to the three piezoelectric material layers 110, respectively, and the multilayered piezoelectric element expands and contracts as a whole due to the piezoelectric effect in the respective piezoelectric material layers 110.

The piezoelectric material layer 110 has a thickness of about 40 μm to 50 μm, for example, and a long side of its bottom surface of about 3 mm to 4 mm, for example. The piezoelectric material layer 110 is formed using a piezoelectric material such as PZT (Pb(lead) zirconate titanate).

Each of the first and second internal electrodes 111 and 112 has a thickness of about 1 μm to 3 μm, for example and may be formed of one kind of material or may have a multilayer structure formed of plural different materials. In the former example, a metal material such as platinum (PT) or silver palladium (Ag—Pd) is used. Further, in the latter example, a two-layer structure including an adhesion layer formed in a thickness of about 50 nm using titanium oxide (TiO2) and a conducting layer formed in a thickness of about 3 μm using platinum (Pt) is used.

The side insulating films 113a and 113b are formed of a highly insulating resin such as an epoxy, silicone, urethane acrylate, or oxetane resin, for example. In such a resin, the Young's modulus is 1.3×10⁹ Pa to 2.0×10⁹ Pa, which is much smaller than that of glass or the like. Accordingly, when the piezoelectric material layers 10 are expanding or contracting, the side insulating films 113a and 113b can follow the expansion and contraction (deformation) of the piezoelectric material layers 10, and thus, there is little braking of the deformation of the piezoelectric material layers 110 due to side insulating films 113a and 113b.

As the side electrodes 114 and 115 and the lower electrode 116 and the upper electrode 117, electrodes of one kind of material selected from gold (Au), platinum (Pt), titanium (Ti), and so on, for example, two-layer structure electrodes of chromium (Cr) and gold (Au), or three-layer structure electrodes of nickel (Ni), titanium (Ti), and platinum (Pt) are used.

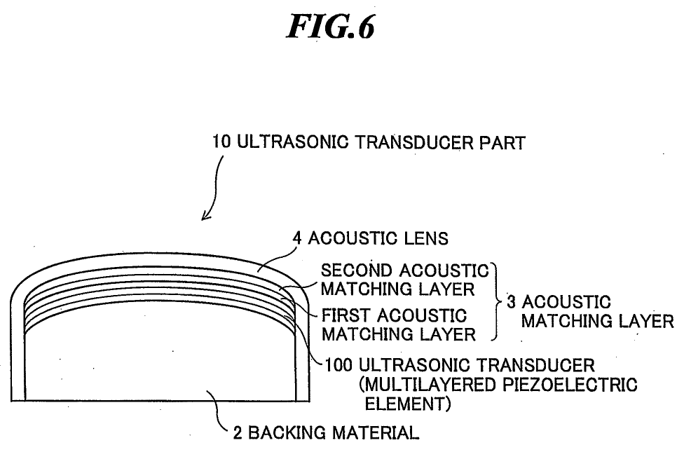

FIG. 6 is a sectional view showing an ultrasonic transducer part 10 to which the multilayered piezoelectric element 100 shown in FIG. 5 is applied. The ultrasonic transducer part 10 has ultrasonic transducers 100 including plural multilayered piezoelectric elements for transmitting and receiving ultrasonic waves, a backing material 2 for supporting the ultrasonic transducers 100, a multilayered acoustic matching layer 3 for providing correct match of acoustic impedances between the ultrasonic transducers 100 and the object, and an acoustic lens 4 for focusing ultrasonic waves in an elevation direction perpendicular to the arrangement direction (azimuth direction) of the ultrasonic transducers 1. The number of the piezoelectric material layers may be two or four or more.

Furthermore, a first signal line holding part 6 and a second signal line holding part 7 for holding plural signal lines (a group of shield lines) for transmitting signals between the plural ultrasonic transducers 1 and the ultrasonic endoscopic apparatus main body are formed in the insertion part of the ultrasonic endoscope. The group of shield lines are electrically connected to the plural ultrasonic transducers 1 via plural signal terminals and guided to the operation part side through the first signal line holding part 6 and the second signal line holding part 7.

In the ultrasonic endoscope according to the embodiment, the first signal line holding part 6 located under the backing material 2 is filled with a highly heat conducting filler and a highly heat conducting layer 11-13 are provided on the bottom surface, side surface, and rear surface of the first signal line holding part 6. As the highly heat conducting filler, for example, a highly heat conducting resin such as a silicone resin or rubber is used. Further, the highly heat conducting layer 11-13 include metal foils (copper foils or the like), graphite sheets, or metal plating layers (copper plating layers or the like). As below, the case of using a highly heat conducting resin as the highly heat conducting filler will be explained.

The first signal line holding part 6 is filled with the highly heat conducting resin together with the group of shield lines drawn from the plural signal terminals provided on the back of the backing material 2. That is, the highly heat conducting resin occupies a region (space) except the signal lines within the region (space) enclosed by the first signal line holding part 6. Therefore, the highly heat conducting resin adheres and thermally coupled to the back of the backing material 2 and the group of shield lines, and thereby, can absorb the heat generated in the ultrasonic transducer part 10.

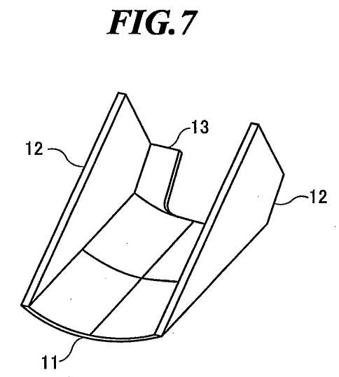

FIG. 7 is a perspective view showing a highly heat conducting layer in the first embodiment of the present invention. The highly heat conducting layer includes a bottom highly heat conducting layer 11, side highly heat conducting layers 12, and rear highly heat conducting layers 13. The bottom highly heat conducting layer 11 is provided on the inner side of the exterior material 8 along the bottom surface of the first signal line holding part 6 opposite to the ultrasonic transducer part 10, and has a shape rounded along the tubular curved surface at the leading end. The side highly heat conducting layers 12 are provided on the inner side of the exterior material 8 (FIG. 3) along the two side surfaces sandwiching a bottom surface of the first signal line holding part 6, and have flat shapes.

The rear highly heat conducting layers 13 are provided at the boundary of the first signal line holding part 6 at the operation part side and plate-like materials that block the surfaces other than the passage opening of the signal lines (the group of tied shield lines). The passage opening of the signal lines serves to tie the group of shield lines and holds them in a stable condition. In this example, the rear highly heat conducting layers 13 are provided at an angle of about 60 degrees tilted from the surface orthogonal to the bottom highly heat conducting layer 11 to the rear side. The bottom highly heat conducting layer 11, the side highly heat conducting layers 12, and the rear highly heat conducting layers 13 are thermally coupled to one another, respectively, and further, thermally coupled to the highly heat conducting resin because these highly heat conducting layers are provided in contact with the highly heat conducting resin.

The highly heat conducting resin has a high coefficient of thermal conductivity of 2 W/mK or more, for example, and the highly heat conducting layer 11-13 have a high coefficient of thermal conductivity of 25 W/mK or more, for example. Further, the thickness of the highly heat conducting layer is effectively 15 μm or more, and preferably about 30 μm to 150 μm for the graphite sheets or copper foils.

Regarding the highly heat conducting layer, not limited to the shown shapes, the bottom highly heat conducting layer 11 may have a flat shape. Further, the side highly heat conducting layers 12 may have shapes rounded along the tubular curved surface at the leading end. Furthermore, the rear highly heat conducting layers 13 may be orthogonal to the bottom highly heat conducting layer 11 at the tilt angle of 0 degree. The rear highly heat conducting layers 13 may be provided at the tilt angle of 0 degree to 80 degrees in a range that the layer can tie the group of shield lines.

According to the above-mentioned configuration, the heat generated in the ultrasonic transducer part 10 is diffused to the filler via the back of the backing material 2 and the shield lines and further diffused to the surface of the exterior material by the highly heat conducting layer, and thereby, the heat can be efficiently released. In order to obtain the high heat dissipation effect despite of the small spatially occupied volume, the thin highly heat conducting layer is used for efficient heat release from the small-diameter endoscope. Further, the heat can be diffused more effectively by providing the highly heat conducting layer inside a casing cooled from the outside.

Next, modified examples of the first embodiment of the present invention will be explained.

In the first embodiment of the present invention or other embodiments, the side highly heat conducting layers 12 or rear highly heat conducting layers 13 shown in FIG. 7 may be omitted.

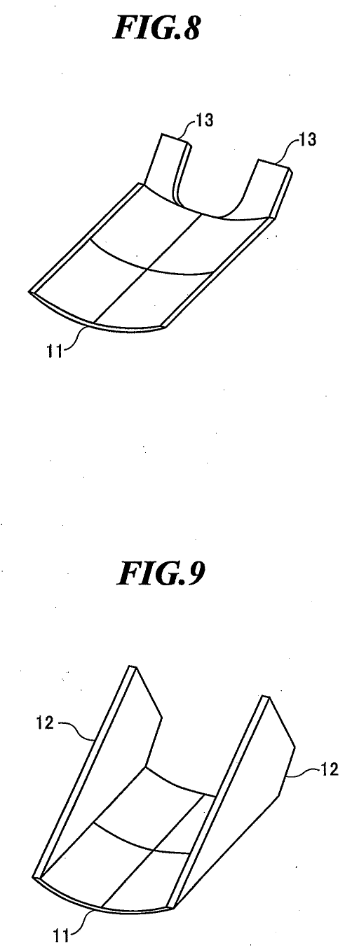

FIG. 8 shows a first modified example of the first embodiment of the present invention. As shown in FIG. 8, in the first modified example, the side highly heat conducting layers are omitted and the bottom highly heat conducting layer 11 and the rear highly heat conducting layers 13 are provided.

FIG. 9 shows a second modified example of the first embodiment of the present invention. As shown in FIG. 9, in the second modified example, the rear highly heat conducting layers are omitted and the bottom highly heat conducting layer 11 and the side highly heat conducting layers 12 are provided.

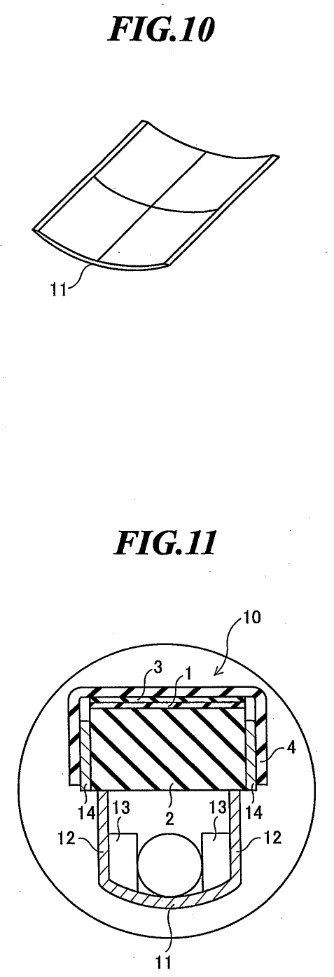

FIG. 10 shows a third modified example of the first embodiment of the present invention. As shown in FIG. 10, in the third modified example, the side highly heat conducting layers and the rear highly heat conducting layers are omitted and only the bottom highly heat conducting layer 11 is provided.

Next, the second embodiment of the present invention will be explained. In the second embodiment, an example in which shield foils are provided on the side surfaces of the ultrasonic transducer part 10 will be explained.

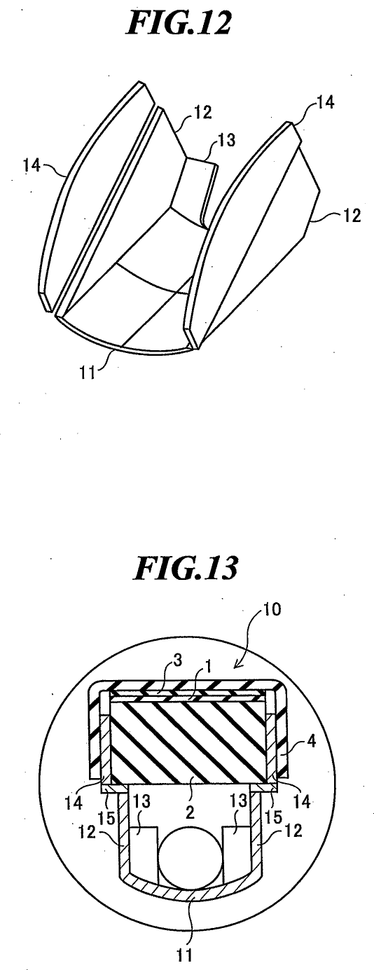

The side section of the leading end of the insertion part of an ultrasonic endoscope according to the second embodiment is the same as that in the first embodiment shown in FIG. 3. FIG. 11 is a front sectional view of the leading end of the insertion part of the ultrasonic endoscope according to the second embodiment of the present invention. Further, FIG. 12 is a perspective view showing a highly heat conducting layer and shield foils in the second embodiment of the present invention.

As shown in FIG. 11, two shield foils 14 are provided along the two side surfaces of the ultrasonic transducer part 10, respectively. The shield foils 14 are formed of copper foils, for example. Note that, as shown in FIGS. 11 and 12, the shield foils 14 are not connected to the side highly heat conducting layers 12. In this embodiment, as is the case of the first embodiment, the first signal line holding part 6 located under the backing material 2 is filled with the highly heat conducting resin and the highly heat conducting layer is provided at least on the bottom surface and the side surfaces of the first signal line holding part 6.

Next, the third embodiment of the present invention will be explained. In the third embodiment, an example in which the shield foils 14 provided on the side surfaces of the ultrasonic transducer part 10 are connected to the side highly heat conducting layers 12 will be explained.

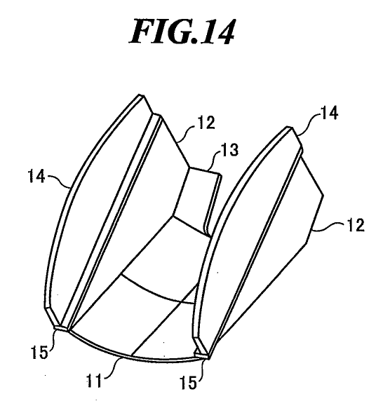

The side section of the leading end of the insertion part of the ultrasonic endoscope according to the third embodiment is the same as that in the first embodiment shown in FIG. 3. FIG. 13 is a front sectional view of the leading end of the insertion part of the ultrasonic endoscope according to the third embodiment of the present invention. Further, FIG. 14 is a perspective view showing a highly heat conducting layer and shield foils in the third embodiment of the present invention.

As shown in FIG. 13, two shield foils 14 are provided along the two side surfaces of the ultrasonic transducer part 10, respectively. As is the case of the second embodiment, the shield foils 14 are formed of copper foils, for example. As shown in FIGS. 13 and 14, the shield foils 14 are connected and thermally coupled to the side highly heat conducting layers 12 via joint foils 15. The joint foils 15 may be formed integrally with the side highly heat conducting layers 12 or shield foils 14, or the side highly heat conducting layers 12, the shield foils 14, and the joint foils 15 may integrally be formed.

In the embodiment, as is the case of the first embodiment, the first signal line holding part 6 located under the backing material 2 is filled with the highly heat conducting resin and the highly heat conducting layer is provided at least on the bottom surface and the side surfaces of the first signal line holding part 6. According to the embodiment, heat diffusion from the shield foils 14 to the side highly heat conducting layers 12 is realized, and thereby, the heat generated in the ultrasonic transducer part 10 is diffused to the bottom highly heat conducting layer 11 and the side highly heat conducting layers 12 via the side surfaces of the backing material 2 and the heat diffusion efficiency as a whole can be further improved.

Next, the fourth embodiment of the present invention will be explained. In the fourth embodiment, an example in which the bottom highly heat conducting layer 11 provided on the bottom surface of the first signal line holding part 6 shown in FIG. 3 is extended toward the operation part beyond the rear highly heat conducting layers 13 will be explained.

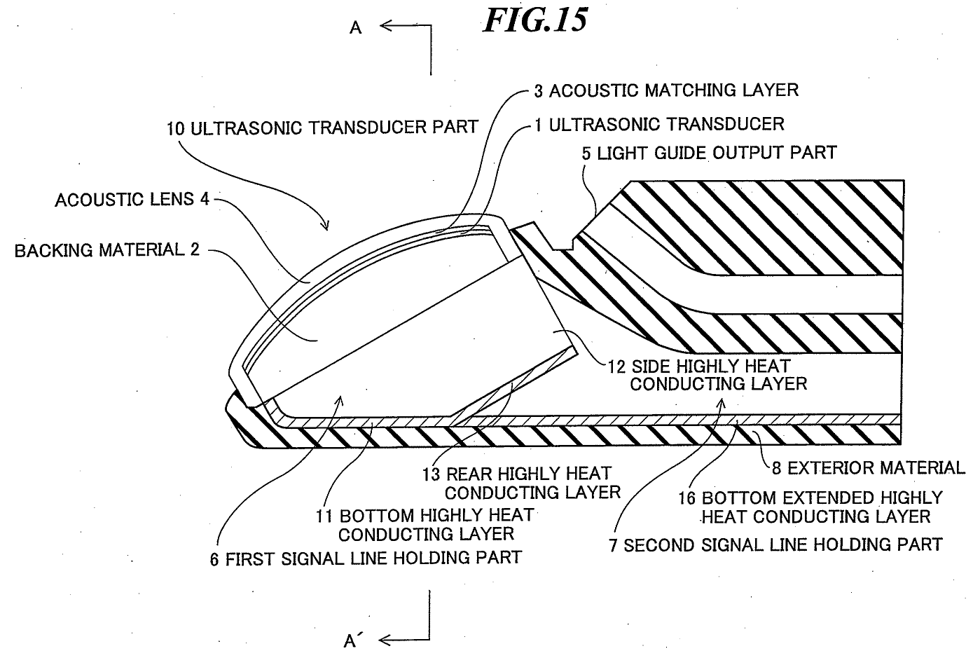

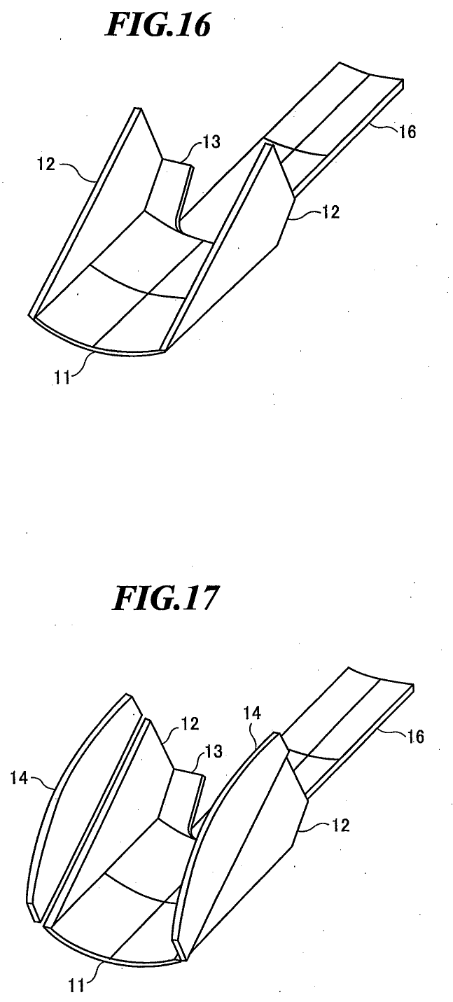

FIG. 15 is a side sectional view schematically showing the leading end of the insertion part of the ultrasonic endoscope according to the fourth embodiment of the present invention. The front section of the leading end of the insertion part of the ultrasonic endoscope according to the fourth embodiment is the same as that in the first embodiment shown in FIG. 4. FIG. 16 is a perspective view showing a highly heat conducting layer in the fourth embodiment of the present invention.

As shown in FIGS. 15 and 16, the ultrasonic endoscope includes a bottom extended highly heat conducting layer 16 formed by extending the bottom highly heat conducting layer 11 provided on the bottom surface of the first signal line holding part 6 beyond the location of the rear highly heat conducting layers 13 toward the operation part side. That is, in the embodiment, the highly heat conducting layer is provided on the bottom surface of the second signal line holding part 7 as well for connection to the highly heat conducting layer on the bottom surface of the first signal line holding part 6. In this example, the bottom extended highly heat conducting layer 16 has a width of about 60% relative to the width of the bottom highly heat conducting layer 11 and a length of about 150% of the length of the bottom highly heat conducting layer 11. Note that, not limited to the example, the width and length may arbitrarily be set. For example, the bottom extended highly heat conducting layer 16 may be provided to the part before the flexing part.

In the embodiment, as is the case of the first embodiment, the first signal line holding part 6 located under the backing material 2 is filled with the highly heat conducting resin. Here, the bottom extended highly heat conducting layer 16 is provided, and thereby, the heat is released from the bottom extended highly heat conducting layer 16 toward the surface of the exterior material 8 and the higher heat dissipation efficiency than in the first embodiment can be obtained.

Next, the fifth embodiment of the present invention will be explained. In the fifth embodiment, an example in which, as is the case of the second embodiment, shield foils that are not connected to the highly heat conducting layer are provided on the side surfaces of the ultrasonic transducer part in addition to the configuration of the fourth embodiment will be explained.

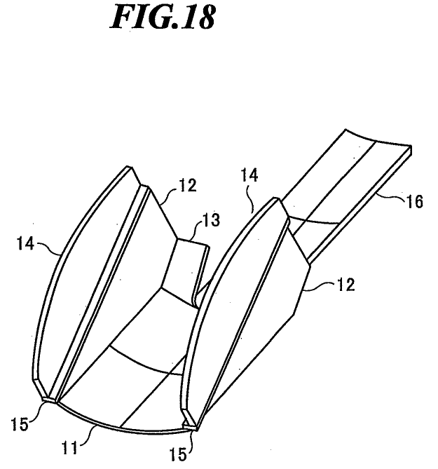

The side section of the leading end of the insertion part of the ultrasonic endoscope according to the fifth embodiment is the same as that in the fourth embodiment shown in FIG. 15. The front section of the leading end of the insertion part of the ultrasonic endoscope according to the fifth embodiment is the same as that in the second embodiment shown in FIG. 11. FIG. 17 is a perspective view showing a highly heat conducting layer and shield foils in the fifth embodiment of the present invention.

In the embodiment, as is the case of the fourth embodiment, the first signal line holding part 6 located under the backing material 2 is filled with the highly heat conducting resin and the highly heat conducting layer is provided at least on the bottom surface and the side surfaces of the first signal line holding part 6. Further, the ultrasonic endoscope includes the bottom extended highly heat conducting layer 16 formed by extending the bottom highly heat conducting layer 11 provided on the bottom surface of the first signal line holding part 6 beyond the location of the rear highly heat conducting layers 13 toward the operation part side and the two shield foils 14 provided along the two side surfaces of the ultrasonic transducer part 10, respectively. According to the embodiment, improvements in heat dissipation by the bottom extended highly heat conducting layer 16 and the shield foils 14 are expected.

Next, the sixth embodiment of the present invention will be explained. In the sixth embodiment, an example in which, as is the case of the third embodiment, shield foils that are connected to the highly heat conducting layer are provided on the side surfaces of the ultrasonic transducer part in addition to the configuration of the fourth embodiment will be explained.

The side section of the leading end of the insertion part of the ultrasonic endoscope according to the sixth embodiment is the same as that in the fourth embodiment shown in FIG. 15. The front section of the leading end of the insertion part of the ultrasonic endoscope according to the sixth embodiment is the same as that in the third embodiment shown in FIG. 13. FIG. 18 is a perspective view showing a highly heat conducting layer and shield foils in the sixth embodiment of the present invention.

In the embodiment, as is the case of the fourth embodiment, the first signal line holding part 6 located under the backing material 2 is filled with the highly heat conducting resin and the highly heat conducting layer is provided at least the bottom surface and the side surfaces of the first signal line holding part 6. Further, the ultrasonic endoscope includes the bottom extended highly heat conducting layer 16 formed by extending the bottom highly heat conducting layer 11 provided on the bottom surface of the first signal line holding part 6 beyond the location of the rear highly heat conducting layers 13 toward the operation part side and the two shield foils 14 provided along the two side surfaces of the ultrasonic transducer part 10, respectively. Since the side highly heat conducting layers 12 are connected to the shield foils 14, the heat is dissipated from the shield foils 14 to the side highly heat conducting layers 12, and heat dissipation via the side highly heat conducting layers 12 by the bottom highly heat conducting layer 11 and the bottom extended highly heat conducting layer 16 can be realized.

Next, the seventh embodiment of the present invention will be explained. In the seventh embodiment, an example in which the side highly heat conducting layers and the rear highly heat conducting layers are omitted and only the bottom highly heat conducting layer and the bottom extended highly heat conducting layer are provided will be explained.

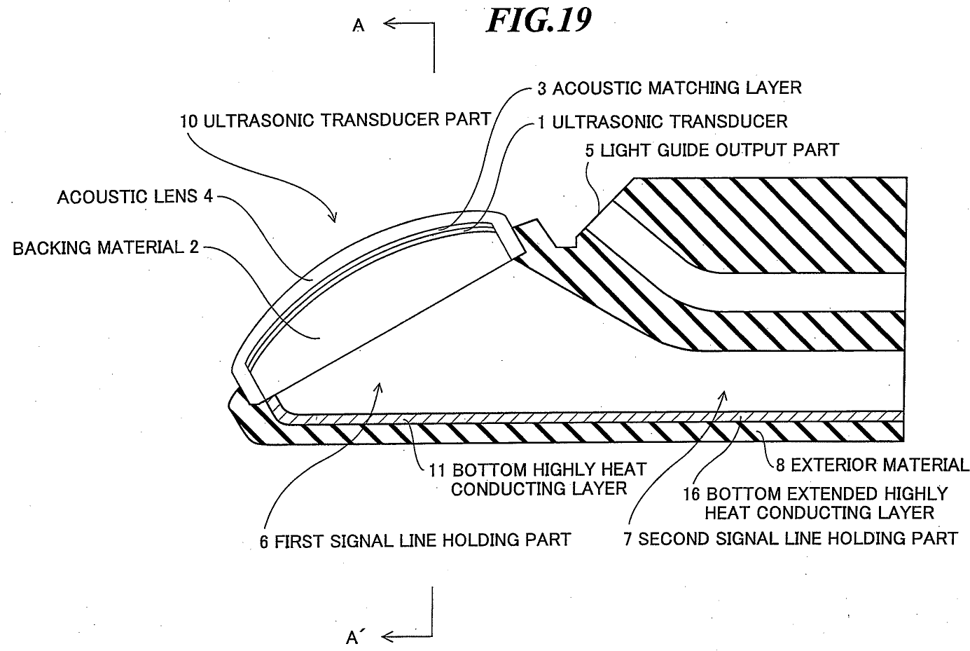

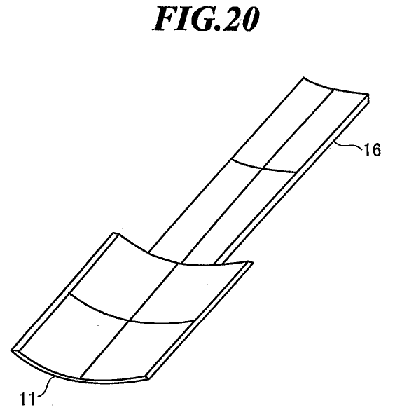

FIG. 19 is a side sectional view schematically showing the leading end of the insertion part of the ultrasonic endoscope according to the seventh embodiment of the present invention. FIG. 20 is a perspective view showing a highly heat conducting layer in the seventh embodiment of the present invention. In the embodiment, the first signal line holding part 6 located under the backing material 2 is filled with the highly heat conducting resin, and heat is released to the exterior material 8 by the bottom highly heat conducting layer 11 and the bottom extended highly heat conducting layer 16.

Next, the eighth embodiment of the present invention will be explained. In the eighth embodiment, an example in which the highly heat conducting layer is thermally coupled to angle rings and/or wires or the like of the endoscope will be explained.

For example, the bottom extended highly heat conducting layer 16 in the fifth embodiment shown in FIG. 17 or the like is thermally coupled to angle rings of the flexing part or thermally coupled to wires provided inside of the covering material of the flexing part and the connecting part. Thereby, heat diffusion can be realized to the angle rings, wires, or the like via the bottom highly heat conducting layer 11 and the bottom extended highly heat conducting layer 16. Further, in the cable containing part of the image pickup device as well, the highly heat conducting resin and/or the highly heat conducting layer are provided around the containing part near the device, and thereby, the heat generated from the image pickup device can be effectively dissipated.

Finally, measurement results of the surface temperature of the ultrasonic endoscope provided with the highly heat conducting filler and the highly heat conducting layer are shown. In the experiment, the surface temperature rise is measured under the condition that the environment temperature is set to 25° C. and the ultrasonic endoscope is left in the air.

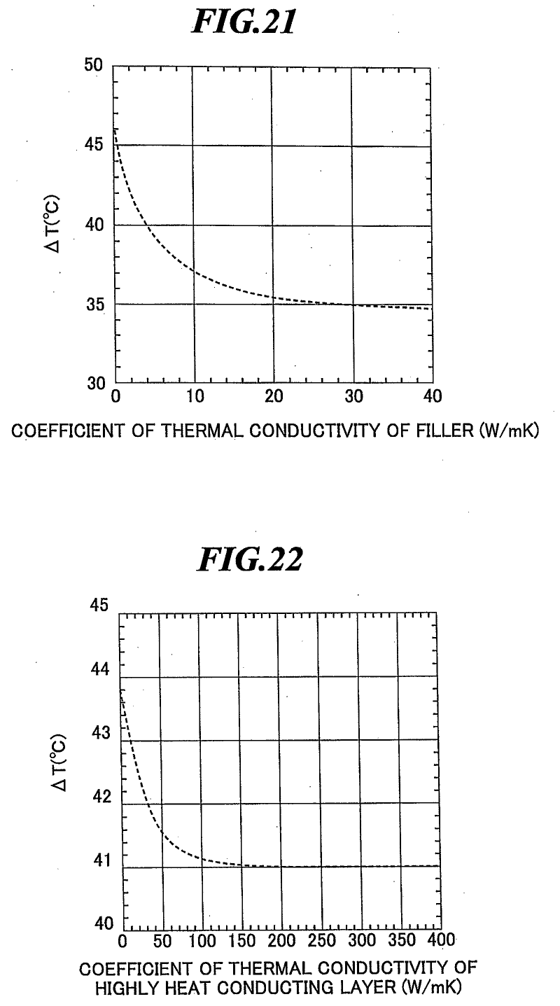

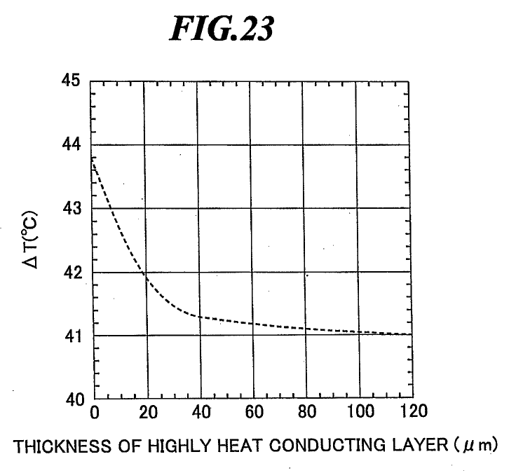

In FIGS. 21-23, the vertical axis indicates the surface temperature rise ΔT (° C.). Further, the horizontal axis indicates the coefficient of thermal conductivity (W/mK) of the filler in FIG. 21, the horizontal axis indicates the coefficient of thermal conductivity (W/mK) of the highly heat conducting layer in FIG. 22, and the horizontal axis indicates the thickness (μm) of the highly heat conducting layer in FIG. 23.

FIG. 21 shows a relationship between the coefficient of thermal conductivity of the filler and the surface temperature rise. Since the coefficient of thermal conductivity of the epoxy resin used as the filler is about 0.45 W/mK, when the filler having a coefficient of thermal conductivity of about 1.5 W/mK is used, temperature drop of about three degrees (7.5%) is realized compared to the case of using the typical epoxy resin.

FIG. 22 shows a relationship between the coefficient of thermal conductivity of the highly heat conducting layer and the surface temperature rise. In the range in which the coefficient of thermal conductivity is smaller than about 70 W/mK, a high correlation is seen between the coefficient of thermal conductivity of the highly heat conducting layer and the surface temperature rise. Therefore, it is known that the heat dissipation effect of the highly heat conducting layer is particularly great if the coefficient of thermal conductivity of the highly heat conducting layer is about 70 W/mK or more. Even if the coefficient of thermal conductivity of the highly heat conducting layer is about 25 W/mK, the heat dissipation effect of the highly heat conducting layer is sufficiently great.

FIG. 23 shows a relationship between the thickness of the highly heat conducting layer and the surface temperature rise. In the range in which the thickness of the highly heat conducting layer is smaller than about 30μm, a high correlation is seen between the thickness of the highly heat conducting layer and the surface temperature rise. Therefore, it is known that the heat dissipation effect of the highly heat conducting layer is particularly great if the thickness of the highly heat conducting layer is about 30 μm or more. Even if the thickness of the highly heat conducting layer is about 15 μm, the heat dissipation effect of the highly heat conducting layer is sufficiently great.

1. An ultrasonic endoscope comprising:
an ultrasonic transducer part including plural ultrasonic transducers for transmitting and receiving ultrasonic waves, and a backing material provided on a back of said plural ultrasonic transducers and having plural signal terminals provided on a surface opposite to said plural ultrasonic transducers;
a signal line holding part including a highly heat conducting filler filling a space holding a group of shield lines electrically connected to said plural ultrasonic transducers via said plural signal terminals, and coupled to said backing material; and
a highly heat conducting layer provided in contact with said signal line holding part, and thereby coupled to said signal line holding part.

2. The ultrasonic endoscope according to claim 1, wherein said highly heat conducting layer is provided on a bottom surface of said signal line holding part opposite to said ultrasonic transducer part and along two side surfaces sandwiching the bottom surface of said signal line holding part.

3. The ultrasonic endoscope according to claim 2, further comprising:
two shield foils respectively provided along the two side surfaces of said ultrasonic transducer part;
wherein said highly heat conducting layer is directly or indirectly connected to said two shield foils.

4. The ultrasonic endoscope according to claim 2, wherein said highly heat conducting layer is formed integrally with two shield foils respectively provided along the two side surfaces of said ultrasonic transducer part.

5. The ultrasonic endoscope according to claim 1, wherein said filler includes one of a silicone resin and a rubber.

6. The ultrasonic endoscope according to claim 1, wherein said highly heat conducting layer includes one of a metal foil, a graphite sheet, and a metal plating layer.

7. The ultrasonic endoscope according to claim 1, wherein said filler has a coefficient of thermal conductivity not less than 1.5 W/mK; and
said highly heat conducting layer has a coefficient of thermal conductivity not less than 25 W/mK.

8. The ultrasonic endoscope according to claim 1, wherein said highly heat conducting layer has a thickness not less than 15 μm.

9. The ultrasonic endoscope according to claim 1, wherein said highly heat conducting layer is provided inside of an exterior material of said ultrasonic endoscope.

10. The ultrasonic endoscope according to claim 9, wherein said exterior material of said ultrasonic endoscope includes a highly heat conducting material.

11. The ultrasonic endoscope according to claim 10, wherein a surface of said highly heat conducting material is coated with a fluorine resin.

12. The ultrasonic endoscope according to claim 1, wherein each of said plural ultrasonic transducers includes a multilayered piezoelectric element.
